(19) United States
(12) Patent Application Publication
(10) Pub. No.: US 2011/0104262 A1
(43) Pub. Date: May 5, 2011

(54) Topical Combinations Comprising an Antimycotic Agent and an Antiviral Agent

(75) Inventors: Amar Lulla, Mumbai (IN); Geena Malhotra, Mumbai (IN)

(73) Assignee: CIPLA LIMITED, Mumbai, Maharashtra (IN)

(21) Appl. No.: 12/988,155

(22) PCT Filed: Apr. 16, 2009
(86) PCT No.: PCT/GB2009/000975

§ 371 Date: Dec. 13, 2010

(30) Foreign Application Priority Data
Apr. 16, 2008 (IN) 862/MUM/2008

(51) Int. Cl. A61K 9/127; A61K 31/496; A61K 31/4196; A61K 31/427; A61K 31/506; A61K 38/12; A61K 31/4418; A61K 31/675; A61K 31/522; A61K 9/00; A61P 31/10; A61P 31/12

(52) U.S. Cl. 424/450; 514/254.07; 514/383; 514/365; 514/256; 514/3.6; 514/345; 514/81; 514/263.38; 424/484; 424/45; 424/43

(57) ABSTRACT

There is provided a pharmaceutical composition comprising an antimycotic agent and an antiviral agent, useful for prophylaxis and/or treatment of an associated infection and/or disease and a method of manufacturing thereof.

### CROSS-REFERENCE TO RELATED APPLICATIONS

This application is a filing under 35 U.S.C. 371 of International Application No. PCT/GB2009/000975 filed Apr. 16, 2009, entitled “Topical Combinations Comprising an Antimycotic Agent and an Antiviral Agent,” claiming priority of Indian Patent Application No. 862/MUM/2008 filed Apr. 16, 2008, which applications are incorporated by reference herein in their entirety.

### FIELD OF INVENTION

The present invention relates to a pharmaceutical composition comprising an antimycotic agent and an antiviral agent, in particular, for prophylaxis and/or treatment of an associated infection and/or disease and a method of manufacturing thereof.

### BACKGROUND OF THE INVENTION

Sexually transmitted infections (STIs), referring to infections that are most often transmitted by direct sexual contact, remain an increasingly serious worldwide public health problem. These STIs, particularly viral infections, present a public health crisis.

Women are especially at risk as they are more susceptible to infection. Many STIs are asymptomatic and there is a high morbidity rate associated with untreated infections.

Since its recognition in 1981, the acquired immunodeficiency syndrome (AIDS) has become a catastrophic pandemic. The AIDS pandemic is a premiere public health concern. Individuals who are at high risk of HIV/AIDS infection are also at risk of infection by other sexually transmitted pathogens. Similarly, individuals at risk for non-HIV/AIDS sexually transmitted pathogens are also at high risk for HIV/AIDS infection.

Additionally, it is significant to note that women comprise the most rapidly increasing population of the AIDS epidemic. Sexual transmission of HIV/AIDS in women occurs by infected semen being placed into the vagina, rectum, or other orifice. Currently, the only prevention strategy available for HIV/AIDS prevention is by using condoms or abstaining from sexual intercourse.

Clinical pathologies attributable to STIs are profound. STIs cause acute and chronic infections, infertility, and in some cases, cancer. Vaccines, which are costly and time-consuming to develop, are not available for certain STIs such as HIV/AIDS prevention. HIV/AIDS treatment employs therapeutic strategies, such as retrovirus triple therapy (e.g., AZT, DDI, etc.) to lower virus burden. However, the high expense of treatment renders this therapeutic option practically unavailable to populations in developing countries where HIV/AIDS is most prevalent. Indeed, the sum of all available STI/AIDS therapeutics is effective against only a limited number of susceptible pathogens. Furthermore, this limited therapeutic arsenal is largely confined to proprietary formulations, which are costly for the afflicted to procure.

Common vaginal infections also pose an increasingly serious worldwide public health problem and can increase the risk of acquiring HIV/AIDS and other STIs. Vaginal candidiasis is the most common form of vaginitis, occurring more frequently than trichophyton, chlamydia, gonorrhea, or other bacterial infections. It is estimated that 75% of women will experience at least one episode of vulvovaginal candidiasis in their lifetime. 40 to 50% will experience a second episode in their life-time. A much smaller (probably less than 5%), but still significant, number of women will suffer from repeated, often intractable attacks. Candidiasis is known to increase the risk of HIV/AIDS acquisition. Bacterial vaginosis (BV), previously known as nonspecific vaginitis or Gardnerella vaginitis is the most common cause of vaginal discharge. It may be the cause of up to 50% of cases of vaginitis in all women and from 10-30% in pregnant women. BV is not a sexually transmitted disease, although it is sometimes listed as one. However, the risk of contracting the disease increases with multiple sex partners. Although treatment is available for these diseases, methods to prevent them and improved methods of treatment are still needed.

Presently marketed vaginal contraceptive compositions, often containing nonoxynol-9 as an active ingredient, are generally known in the art. While presently marketed vaginal contraceptive formulations aid in preventing pregnancy, their ability to effectively prevent STIs, particularly HIV/AIDS as well as oral, rectal, and vaginal infections, is very limited. Nonoxynol-9 and other detergents as well as their compositions can destroy the natural and safe ecology of the vagina, such as by inactivating lactobacillus bacteria. Further, spermicides may cause vaginal irritation, particularly with frequent exposure or higher doses. Recent analyses show that nonoxynol-9, when used frequently by women at high risk, may increase the risk of HIV infection (WHO 2002, WHO/CONRAD technical consultation on nonoxynol-9, Geneva).

Many antiviral agents have been developed for the treatment of patients with human immunodeficiency virus (HIV) infection. However, only temporary and limited benefits are observed in HIV-infected patients treated with any of the actual anti-retrovirals or combinations of them. The limited ability of these agents to decrease viral burden, the rapid development of resistance and the toxic side-effects of most drugs has limited their long-term efficacy. One major problem associated with the administration of antiviral agents to patients is their poor ability to penetrate and target infected cells. Rapid drug clearance and the toxicity of parent compounds or metabolites constitute also some of the major drawbacks which may slow down the development and use of many antiviral agents. Given the severe toxicity of antiviral agents actually available to treat AIDS and other viral diseases and their limited ability to target infected cells, strategies aimed at reaching therapeutic levels of drugs into infected cells and reducing toxicity should be explored.

U.S. 2005/0037033 discloses a microbicidal compositions containing Ciclopirox olamine for preventing the transmission of or treating sexually transmitted infections and/or common vaginal infections.

WO 96/02226 discloses a pharmaceutical composition comprising a combination of 1-hydroxy-2-pyridones such as Ciclopirox or Octopirox and Crotamiton as an antifungal agent activity enhancer.

WO 97/17075 discloses a topical foamable pharmaceutical composition of Ciclopirox or Ciclopirox olamine and surfactant for treating skin diseases induced by oval Pityrosporum.

U.S. 2005/0196418 discloses a composition comprising a molecular complex formed between an alkaline pharmaceutical drug and at least one selected from a hydroxyacid, a polyhydroxy acid, a related acid, a lactone, or combinations thereof.

U.S. 2005/0276836 discloses a vaginal device for delivering therapeutic and/or health-promoting agents, wherein said vaginal device is a vaginal tampon, vaginal tampon-like device, vaginal ring and others.

U.S. Pat. Nos. 4,108,309; 4,360,013; and 4,589,880 disclose suitable devices for applying the composition to the vagina.

U.S. Pat. No. 5,292,516 discloses a process of treating a condition by means of application of in-situ gel formulations comprising poloxamers administered by means of medical device.

However, use of such delivery devices for applying compositions to the vagina may cause an internal harm, for example, rashes or bleeding.

There still remains a need to develop a medicament and/or formulation which stand against a multitude of resistant strains, could protect the drug against enzymatic degradation, improve their pharmacokinetics and tissue distribution, while minimizing disruptions to vaginal ecology and epithelium.

### OBJECT OF THE INVENTION

The object of the present invention is to provide a novel pharmaceutical composition, in particular, for prophylaxis and/or treatment of sexually transmitted infections including HIV/AIDS and/or common vaginal infections which stands against resistant strains.

Another object of the present invention is to provide a novel composition, in particular, for prophylaxis and/or treatment of sexually transmitted infections including HIV/AIDS and/or common vaginal infections while minimizing disruptions to vaginal ecology and epithelium without compromising the stability and efficacy of the formulation.

Yet another object of the present invention is to provide topical liposomal formulations of drugs for the treatment of sexually transmitted infections including HIV/AIDS and/or related vaginal infections that result in an increased efficacy and reduced toxicity of antiviral agents in humans suffering from sexually transmitted infections including HIV and/or related vaginal infections.

Still another object of the present invention is to provide a novel pharmaceutical composition and/or medicament with ease of manufacture.

### SUMMARY OF THE INVENTION

According to one aspect, there is provided a novel pharmaceutical composition for topical administration comprising one or more antimycotics or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and one or more anti-viral agent/s or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and one or more pharmaceutically acceptable excipients.

According to a second aspect, there is provided a novel pharmaceutical composition for topical administration comprising Ciclopirox or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and Tenofovir or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and one or more pharmaceutically acceptable excipients.

According to a third aspect, there is provided a novel pharmaceutical composition for topical administration comprising liposome encapsulated Tenofovir or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof wherein the said liposomes allows high cellular penetration, good in vitro antiviral activity against HIV, which in turn provides a marked improvement of the pharmacokinetics of drugs.

According to a fourth aspect, there is provided a process of manufacturing the said novel pharmaceutical composition for topical administration comprising Ciclopirox or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof; one or more anti-viral agent/s or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and one or more pharmaceutically acceptable excipients.

According to a fifth aspect, there is provided a process of manufacturing the said novel pharmaceutical composition for topical administration comprising liposome encapsulated Tenofovir or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

According to a further aspect, there is provided a novel pharmaceutical composition for topical administration for prophylaxis and/or treatment of sexually transmitted infections including HIV/AIDS and/or related vaginal infections.

Any of the active materials described and claimed in this specification may be provided in the form of the free material or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

### DETAILED DESCRIPTION OF THE INVENTION

The inventors have surprisingly found that by incorporation of liposome en-capsulated Tenofovir in a suitable topical formulation (e.g., gel composition or spray foam) prevented the transmission of infectious pathogens and its permeation through the membrane of mucosa by means of physical cum pharmacological barrier thereby preventing infection of host cells.

It was further found that by topical application of combination of anti-infectives like Ciclopirox and/or Tenofovir resulted better penetration in the fungal and vaginal infections, thereby destroying plasma membrane which in turn prevented or limited contact of the fungus and/or virus or its carrier cells with the epithelium or prevented or hindered its entry into the orifice by forming a physical cum pharmacological barrier and thus preventing recurrence of infection for a considerable period of time.

Liposomes are microscopic vesicles in which a variety of drugs can be incorporated, to form a non-toxic and biodegradable formulation because of the similarity of the primary components of liposomes with natural membranes. It allows high cellular penetration, efficient targeting of macrophage-rich tissues and a marked improvement in drug pharmacokinetics.

The liposomal topical formulation, according to the present invention, provides improved delivery of active agents to the infected cells, and also reduces the toxic effects associated with their administration which in turn improved efficacy and safety of the drug used for the treatment of sexually transmitted infections including HIV/AIDS and/or related vaginal infections.

When applied locally to mucosa or skin, liposomes are usually taken up by monocytes and macrophages and also by Langerhans cells which may capture and harbor HIV. Consequently, in contrast with free drugs, which tend to diffuse rapidly through the mucosa and reach the circulation, the use of drugs within liposomes and incorporated into a topical formulation (e.g., gel formulation or spray foam) concentrates the active agents within infected cells as well as within cells susceptible to HIV infection.

It will be well acknowledged to a person skilled in the art that combination of anti-mycotic (e.g., Ciclopirox) with liposome encapsulated Tenofovir when applied topically; may achieve the same benefits as described above.

According to preferred embodiment, topical combination comprising Ciclopirox and Tenofovir (or liposome encapsulated Tenofovir) exhibited excellent anti-infective activity against opportunistic infections encountered in STI's and/or AIDS, such as vaginal infections without compromising on the stability of the formulation.

According to the present invention, the protection from sexually transmitted infections, such as HIV/AIDS, and common vaginal infections, such as bacterial vaginosis and vaginal candidiasis, may be obtained by application of the novel pharmaceutical composition to vagina, rectum, or other orifice.

According to the present invention, pharmaceutical compositions may be used alone or in conjunction with delivery and/or contraceptive devices or methods, such as mechanical barrier-type devices. Pharmaceutical compositions, according to the present invention, may be formulated in various dosage forms including a base or carrier, such as a foam, cream, wash, gel, suppository, ovule, lotion, ointment, film, foaming tablet, tampon, vaginal spray, or aerosol.

According to the present invention, the novel pharmaceutical composition for topical administration may encompass one or more anti-infectives selected from the class of, but not limited to, antimycotics, antimycobacterials, antibacterials, antivirals or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof.

Accordingly, as a preferred embodiment, a novel pharmaceutical composition for topical administration may comprise antimycotics (e.g., Ciclopirox) or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and Tenofovir (or liposome encapsulated Tenofovir) or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and one or more pharmaceutically acceptable excipients for prophylaxis and/or treatment of STI's including HIV/AIDS and related viral infections.

Alternatively, the novel pharmaceutical composition for topical administration may comprise one or more antimycotics selected from the class of, but not limited to ketoconazole, itraconazole, fluconazole, ravuconazole, posaconazole, voricnazole, caspofungin, hydroxypyridones derivatives such as ciclopirox, mimosine, deferipone or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof with one or more antiviral agents selected from the class of, but not limited to Tenofovir, Acyclovir and Ganciclovir or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and one or more pharmaceutically acceptable excipients.

As a preferable embodiment, the pharmaceutical composition for topical administration may comprise liposome encapsulated Tenofovir with one or more pharmaceutically acceptable excipients or combination of Ciclopirox [or liposome encapsulated Ciclopirox] or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof with Tenofovir (or liposome encapsulated Tenofovir) or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and one or more pharmaceutically acceptable excipients.

According to the present invention, the pharmaceutical formulations may be applied to the body cavities such as the vagina and rectum. It will be readily acknowledged to a person skilled in the art; that the formulation may also be applied to the skin and other mucous membranes. Preferably, the said novel pharmaceutical formulations inactivate bacteria, fungi and/or viruses, and are stable at ambient temperature, compatible and active after mixture with cosmetically acceptable formulations, non-toxic and non-damaging to vulvar, vaginal, cervical, penile, or other epithelium.

The pharmaceutical composition of the present invention prevents the transmission of or treats sexually transmitted infections and/or common vaginal infections. Sexually transmitted infections include, but are not limited to, HIV/AIDS, herpes (caused by herpes simplex virus type 1 (HSV-1) or herpes simplex virus type 2 (HSV-2)), gonorrhea, chlamydia, syphilis, and trichomoniasis. Common vaginal infections include, but are not limited to, bacterial vaginosis (BV) and vaginal candidiasis. Similar compositions and methods of application of such compositions, as described herein, can be used for treating sexually transmitted infections and/or common vaginal infections and for preventing the transmission of sexually transmitted infections and/or common vaginal infections.

Preferably, the present invention involves the topical application of the formulation. In the context of the present invention, it is to be understood that the term topical application includes application to the body cavities as well as to the skin. Thus, in a preferred embodiment, the formulation is applied to a body cavity such as the vagina, anus, rectum, or mouth. In a particularly preferred embodiment, the composition is applied to the vagina.

In a preferred embodiment, the topical application is carried out prior to the beginning of vaginal intercourse, preferably from 0 to 8 hours, more preferably from 0 to 60 minutes. The composition including the combination may be used independent from intercourse.

According to the preferred embodiments, the pharmaceutically acceptable excipients may include, but are not limited to, one or more surfactant, emollient or humectant, pH adjusting agent, fatty alcohol, preservative, lipid core components (e.g., phospholipids), organic solvent, gelling agents, chelating agents, film forming polymers, antioxidants, propellants or combinations thereof based on the type/route of administration of the formulation.

The surfactants may be selected from, but not limited to, Polyoxyethylene alcohol, alkylphenol ethoxylate, polysorbate 80, polysorbate 60, polymethylsiloxane, alkylphenol ethoxylate, poloxomer 407, sorbitan monostearate, sorbitan monolaurate, sorbitan monopalmitate, sorbitan monooleate, polyethylene glycol (PEG) stearic acid esters (e.g., polyethylene glycol 100 stearate).

Suitable humectants and/or emollients provide smoothness and lubricity which, in turn, facilitate the loading and dispensing of the formulation. The emollients and/or humectants may be selected from, but not limited to, polyhydric alcohols such as glycols, and polysaccharides, such as ethylene glycol, propylene glycol, butylene glycol, diethylene glycol, dipropylene glycol, glycerin, diglycerin, sorbitol, malvitol, trehalose, raffinose, xylitol, mannitol, polyethylene glycol, propylene glycol, polyglycerin, cholesterol, squaline, fatty acids, octyldodecanol, myristyl alcohol, urea, lanolin, lactic acid, esters such as isopropyl stearate, isopropyl myristate, isopropyl palmitate, and isopropyl laurate, and the like.

The pH adjusting agents may be selected from, but not limited to, lactic acid, sodium hydroxide, acetic acid, citric acid, tartaric acid, propionic acid, sodium phosphate, ammonia solution, triethanolamine, sodium borate, sodium carbonate, potassium hydroxide, and like.

The fatty alcohols may be selected from, but not limited to, stearyl alcohol, cetyl alcohol, capryl alcohol, myristyl alcohol, 1-dodecanol, palitoleyl alcohol, oleyl alcohol, linoleyl alcohol, isostearyl alcohol and like, preferably stearyl alcohol, and cetyl alcohol.

The preservatives may be selected from, but not limited to, benzyl alcohol, hydroxybenzoates (parabens), Benzoic Acid, Chlorphenesin, Sorbic Acid, Phenoxyethanol and like.

Lipid core components may be selected from, but not limited to, natural phospholipids such as egg yolk lecithin (phosphatidylcholine), soybean lecithin, lysolecithin, sphingomyelin, phosphatidic acid, phosphatidylserine, phosphatidylcholine, phosphatidylglycerol, phosphatidylinositol, phosphatidylethanolamine, diphosphatidylglycerol, cardiolipin, plasmalogen, etc., or hydrogenation products obtainable from said phospholipids by the conventional technology (Hydrogenated soy phosphatidyl choline), and synthetic phospholipids such as dicetyl phosphate, distearoylphosphatidylcholine, dipalmitoylphosphatidylcholine, dipalmitoylphosphatidyl glycerol, distearoylphosphatidyl glycerol, dilaurylphosphatidylglycerol, dipalmitoylphosphatidylethanolamine, dipalmitoylphosphatidylserine, eleostearoylphosphatidylcholine, eleostearoylphosphatidylethanolamine, eleostearoylphosphatidylserine, dipalmitoylphosphatidyl acid, dipalmitoylphosphatidyl ethanolamine, their salts and the corresponding distearoyl- and dimyristyl-counterparts and/or mixtures thereof.

These lipids or their mixtures may further contain substances selected from dicetylphosphate, cholesterol, coprostanol, cholestanol, cholestane, ergosterol, phytosterol, sitosterol, lanosterol, protein (e.g., albumin, immunoglobulin, casein, insulin, hemoglobin, lysozyme, immunoglobulin, [alpha]-2-macroglobulin, fibronectin, vitronectin, fibrinogen, lipase, or enzyme) which strengthens the lipid and other additives like α-tocopherol, stearic acid, antioxidants, BHT (butylhydroxytoluene), ascorbic acid, deferoxime mesylate, stearyl amine and/or mixtures thereof.

Alternatively, gelling agents such as, alginic acid, sodium alginate, potassium alginate, agar, carrageenan, pectin, gelatin, calcium alginate, carbomers, methyl cellulose, sodium carboxy methyl cellulose and other cellulose derivatives, carbopol, bentonite (preferably carbomers) may be used in combination with bioadhesives which includes, but not limited to, gelatin, carbopol 934, polycarbophil, cross-linked polymethacrylic acid, hydroxypropyl methyl cellulose, ethyl cellulose, preferably carbopol and methyl cellulose.

The chelating agents may be selected from, but not limited to, disodium edetate, sodium citrate, condensed sodium phosphate, diethylenetriamine penta-acetic acid and like.

Film forming polymers may be selected from, but are not limited to, carbomers such as carboxymethylene polymers including acrylic acid polymers, and acrylic acid copolymers, acrylic acid alkyl ester monomers, maleic acid alkyl esters, crotonic acid alkyl ester monomers, vinyl ester monomers, cellulose derivatives, vinylpyrrolidone-vinyl acetate copolymers, polyurethane, preferably carbopol, hydroxyethyl cellulose, methyl cellulose, vinylpyrrolidone-vinyl acetate copolymers.

Antioxidants may be selected from but are not limited to, ascorbate, BHT, BHA, sodium metabisulphite, alpha-tocopherol or its synthetic derivatives, EDTA and like.

Propellants may be selected from volatile hydrocarbons such as butane, propane, isobutane and fluorocarbon gases or mixtures thereof, fluorohydrocarbon (HFCs) propellants such as 1,1,1,2-tetrafluorethane, and 1,1,1,2,3,3,3-heptafluoropropane, 1,1-difluoro ethane and 1,1,1,3,3,3-hexafluoropropane, preferably HFC 134a or HFA 227.

According to a first preferred embodiment of the present invention, the topical pharmaceutical gel formulation comprise Ciclopirox (or liposomal encapsulated Ciclopirox) or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and Tenofovir (or liposomal encapsulated Tenofovir) or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and one or more pharmaceutically acceptable excipients such as gelling agents preferably carbopol and/or cellulose derivatives; lipid core preferably egg lecithin or soya lecithin; organic solvent preferably ethanol; preservatives and pH adjusting agents.

According to a second preferred embodiment of the present invention, the topical pharmaceutical formulation in the form of spray-foam comprise ciclopirox or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and Tenofovir (or liposomal encapsulated Tenofovir) or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and one or more pharmaceutically acceptable excipients such as fatty alcohols preferably cetyl alcohol and stearyl alcohol; humectants preferably glycerin; surfactants preferably polyethylene glycol; emollients such as propylene glycol and propellant preferably hydrofluorocarbon [HFC-134].

According to a third preferred embodiment of the present invention, the topical pharmaceutical gel formulation comprises liposome encapsulated Tenofovir or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and one or more pharmaceutically acceptable excipients and one or more pharmaceutically acceptable excipients such as gelling agents preferably carbopol or cellulose derivatives; lipid core preferably egg lecithin or soya lecithin; organic solvent preferably ethanol; preservatives and pH adjusting agents

According to a fourth preferred embodiment of the present invention, the topical pharmaceutical formulation in the form of spray-foam comprises liposome encapsulated Tenofovir or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof and Ciclopirox [or liposome encapsulated Ciclopirox] or its pharmaceutically acceptable salts, pharmaceutically acceptable solvates, pharmaceutically acceptable enantiomers, pharmaceutically acceptable derivatives, pharmaceutically acceptable polymorphs or pharmaceutically acceptable prodrugs thereof one or more pharmaceutically acceptable excipients and one or more pharmaceutically acceptable excipients such as lipid core preferably egg lecithin or soya lecithin; film forming polymer preferably Kollidon VA64 and organic solvent such as ethanol.

According to the present invention, there is further provided process/method(s) of preparing the said pharmaceutical composition.

According to the above mentioned embodiment, there is provided a method of manufacturing the said topical pharmaceutical liposomal gel comprising:
(a) Dissolving an antiviral agent in a lipid core component with suitable organic solvent to obtain a solution;
(b) Homogenizing the solution of step (a) solution with water;
(c) Introducing an antimycotic agent, a film forming polymer and a preservative in water, followed by pH adjustment, to form a slurry; and
(d) Forming a liposomal gel by adding and stiffing the homogenized solution with the slurry.

According to the second embodiment, there is provided a method of manufacturing the said topical pharmaceutical foam comprising:
(a) Dissolving a fatty alcohol and a surfactant in a suitable organic solvent to form a solution;
(b) Adding an antimycotic agent, an antiviral agent and an emollient/humectant to the solution; and
(c) Filling the aerosol foam in aluminium canisters and pressurizing with propellants.

According to the third embodiment, there is provided a method of manufacturing the said topical liposomal spray comprising:
(a) Dissolving the actives in lipid component along with soya lecithin and Kollidon V A 64 in a suitable organic solvent; and
(b) Filling the above solution in aluminium canisters and pressurizing with propellant.

According to the fourth embodiment, there is provided a method of manufacturing the said topical liposomal foam wherein the actives and lipid component were dissolved with other excipients in suitable organic solvent.

According to the intended therapeutic purpose, the composition according to the present invention may be formulated into pharmaceutical preparations common in the pharmaceutical field, which include, gel; a spray; a foam; a cream; a wash; a pessary; an ovule; a lotion; an ointment; a film; a foaming tablet; a tampon; a vaginal spray; solution; a bath; a liniment; a patch; a pad; a bandage.

According to present invention, suitable excipients required for the formulation of the above mentioned dosage forms may be used.

Alternatively, for ointment formulation, taking into consideration various factors including temperature of the skin surface, pH of the skin, transdermal water loss levels and total lipid levels of the epidermis, the present composition may be mixed with oleaginous bases, which are exemplified by Vaseline, liquid paraffin, paraffin, plastibase, silicon, lard, vegetable oils, waxes and purified lanolin, water-soluble bases, emulsion bases, suspension bases, and the like. The ointments may be supplemented with an antioxidant (e.g., tocoperol, BHA, BHT, NDGA, etc.), an antiseptic (e.g., phenolic compounds, chlorobutanol, benzylalcohol, parabens, benzoic acid, etc.), a humectant (e.g., glycerin, propylene glycol, sorbitol, etc.), a solution adjuvant (e.g., ethanol, propylene glycol, etc.), a softening adjuvant (e.g., liquid paraffin, glycerin, propylene glycol, surfactants, etc.), and other additives.

Alternatively, for spray formulation, the additives may be mixed with a propellant to disperse a water-dispersed concentrate or humidified powder. For patch formulation, a permeation stimulator may be used to increase the permeation of compounds through the skin.

The pH of the composition of the invention can be physiologically compatible and/or sufficient to maintain stability of the composition. According to preferred embodiments, the composition of the present invention has a pH range of 4.0 to 6.0.

The present invention further provides for a method of prophylaxis and/or treatment of sexually transmitted infections including HIV/AIDS and/or common vaginal infections by application and/or use of a therapeutically effective amount of the combination in a suitable pharmaceutical composition of the present invention to a mammal in need thereof.

### EXAMPLES

The following examples are for the purpose of illustration of the invention only and are not intended in any way to limit the scope of the present invention.

Ciclopirox and Tenofovir Vaginal Liposomal Gel:

### Formula:

| Sr. No. | Ingredients | Qty (% w/w) |
|---|---|---|
| 1. | Ciclopirox olamine | 1.00 |
| 2. | Tenofovir disoproxil fumarate | 1.00 |
| 3. | Lecithin (Egg/Soya) | 1.0-30.0 |
| 4. | Ethanol | 1.0-50.0 |
| 5. | Hydroxyethylcellulose/ | 0.1-4.0 |
| | Methyl cellulose | |
| 6. | Methyl Paraben | 0.05-0.3 |
| 7. | Propyl Paraben | 0.005-0.05 |
| 8. | Triethanolamine | q.s. to adjust pH |
| 9. | Water | q.s. to 100% |

### Process:

(a) The antiviral agent was dissolved in lipid component with suitable organic solvent and homogenized;
(b) The antimycotic agent was dissolved in lipid component with suitable organic solvent and homogenized;
(c) Slurry of film forming polymers and preservatives was made in water followed by pH adjustment.
(d) Finally, liposomal gel was formed by adding and stiffing homogenized solutions of steps (a) and (b) with slurry.

Ciclopirox and Tenofovir Vaginal Liposomal Gel:

### Formula:

| Sr. No. | Ingredients | Qty (% w/w) |
|---|---|---|
| 1. | Ciclopirox olamine | 1.00 |
| 2. | Tenofovir disoproxil fumarate | 1.00 |
| 3. | Lecithin (Egg/Soya) | 1.0-30.0 |
| 4. | Ethanol | 1.0-50.0 |
| 5. | Hydroxyethylcellulose/ | 0.1-4.0 |
| | Methyl cellulose | |
| 6. | Methyl Paraben | 0.05-0.3 |
| 7. | Propyl Paraben | 0.005-0.05 |
| 8. | Triethanolamine | q.s. to adjust pH |
| 9. | Water | q.s. to 100% |

### Process:

(a) The antiviral agent is dissolved in lipid component with suitable organic solvent.
(b) The above solution was then homogenized in water.
(c) Slurry of antimycotic agent, film forming polymers and preservatives was made in water followed by pH adjustment.
(d) Finally, liposomal gel was formed by adding and stiffing homogenized solution with slurry.

Ciclopirox and Tenofovir Vaginal Foam:

| Sr. No. | Ingredients | Qty (% w/w) |
|---|---|---|
| 1. | Ciclopirox olamine | 1.00 |
| 2. | Tenofovir disoproxil | 1.00 |
| | fumarate | |
| 3. | Cetyl alcohol | 2.00 |
| 4. | Stearyl alcohol | 1.00 |
| 5. | Glycerin | 35.00 |
| 6. | Polyethylene glycol- | 1.00 |
| | 100 stearate | |
| 7. | Ethanol | 25.00 |
| 8. | Propylene glycol | 33.88 |
| 9. | Propellant: | 10.0-25.0 |
| | Hydrofluorocarbon | |
| | (HFC-134a) INH | |

### Process:

(1) Cetyl alcohol, stearyl alcohol and Polyethylene glycol-100 stearate was dissolved in ethanol.
(2) To the above solution, Ciclopirox and Tenofovir were added.
(3) Propylene glycol and Glycerin were then added to the solution obtained in step (2) and mixed.
(4) Finally, the above solution was filled in aluminium canisters and pressurized with propellant.

Tenofovir Liposomal Gel:

| Sr. No. | Ingredients | Qty (% w/w) |
|---|---|---|
| 1. | Tenofovir disoproxil | 1.00 |
| | fumarate | |
| 2. | Ciclopirox olamine | 1.00 |
| 3. | Lecithin (Egg/Soya) | 10.00 |
| 4. | Ethanol | 20.00 |
| 5. | Carbopol/Methyl | 1.00 |
| | cellulose | |
| 6. | Methyl Paraben | 0.50 |
| 7. | Propyl Paraben | 0.05 |
| 8. | Triethanolamine | q.s. to adjust pH |
| 9. | Water | q.s. to 100.00 |

### Procedure:

(1) Tenofovir disoproxil fumarate and Lecithin were dissolved in Ethanol.
(2) The above solution was the added in water under ultraturrax and Homogenized for 20 min.
(3) Slurry of Carbopol or Methyl cellulose in water was made containing dissolved methyl and propyl paraben.
(4) pH was adjusted with Triethanol amine, if required.
(5) The homogenize blend was added in Carbopol or Methyl cellulose slurry. Finally it was stirred for 30 min to form a liposomal gel.

Tenofovir Liposomal Spray:

| Sr. No. | Ingredients | Qty (% w/w) |
|---|---|---|
| 1. | Tenofovir disoproxil | 1.00 |
| | fumarate | |
| 2. | Ciclopirox olamine | 1.00 |
| 3. | Lecithin (Egg/Soya) | 10.00 |
| 4. | Kollidon VA64 | 2.50 |
| 5. | Ethanol | q.s. to 100.00 |

### Procedure:

(1) Tenofovir disoproxil fumarate, N-vinylpyrrolidone-vinyl acetate copolymer were dissolved in ethanol.
(2) Ciclopirox olamine, N-vinylpyrrolidone-vinyl acetate copolymer were dissolved in ethanol.
(3) The above solutions were combined and filled in aluminium canisters and pressurized with propellant.

Tenofovir Liposomal Spray:

| Sr. No. | Ingredients | Qty (% w/w) |
|---|---|---|
| 1. | Tenofovir disoproxil | 1.00 |
| | fumarate | |
| 2. | Ciclopirox olamine | 1.00 |
| 3. | Lecithin (Egg/Soya) | 10.00 |
| 4. | Kollidon VA64 | 2.50 |
| 5. | Ethanol | q.s. to 100.00 |

### Procedure:

(1) Tenofovir disoproxil fumarate, N-vinylpyrrolidone-vinyl acetate copolymer were dissolved in ethanol and then ciclopirox olamine was added.
(2) The above solution was filled in aluminium canisters and pressurized with propellant.

It will be readily apparent to one skilled in the art that varying substitutions and modifications may be made to the invention disclosed herein without departing from the spirit of the invention. Thus, it should be understood that although the present invention has been specifically disclosed by the preferred embodiments and optional features, modification and variation of the concepts herein disclosed may be resorted to by those skilled in the art, and such modifications and variations are considered to be falling within the scope of the invention.

It is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It must be noted that, as used in this specification and the appended claims, the singular forms “a,” “an” and “the” include plural references unless the context clearly dictates otherwise. Thus, for example, reference to “a diluent” includes a single diluent as well as two or more different diluents, reference to a “disintegrant” refers to a single disintegrant or combination of two or more disintegrants, and the like.

What is claimed is:

1. A pharmaceutical composition comprising an antimycotic agent and an antiviral agent, optionally in combination with one or more pharmaceutically acceptable excipients.

2. The pharmaceutical composition according to claim 1, wherein the antimycotic agent is ketoconazole; itraconazole; fluconazole; ravuconazole; posaconazole; voricnazole; caspofungin; or a hydroxypyridone derivative, such as ciclopirox, mimosine, or deferipone and the antiviral agent is Tenofovir, Acyclovir and/or Ganciclovir.

3. The pharmaceutical composition according to claim 1, wherein the antimycotic agent is Ciclopirox and the antiviral agent is Tenofovir.

4. The pharmaceutical composition according to claim 1, which is in the form of a gel; a spray; a foam; a cream; a wash; a pessary; an ovule; a lotion; an ointment; a film; a foaming tablet; a tampon; a vaginal spray; solution; a bath; a liniment; a patch; a pad; or a bandage.

5. The pharmaceutical composition according to claim 4, wherein the composition is in the form of a gel or spray.

6. The pharmaceutical composition according to claim 5 comprising liposomes and wherein the antiviral agent and/or the antimycotic agent is encapsulated in the liposomes.

7. The pharmaceutical composition according to claim 6, wherein the liposomes comprise: a natural phospholipid such as egg yolk lecithin (phosphatidylcholine), soybean lecithin, lysolecithin, sphingomyelin, phosphatidic acid, phosphatidylserine, phosphatidylcholine, phosphatidylglycerol, phosphatidylinositol, phosphatidylethanolamine, diphosphatidylglycerol, cardiolipin, plasmalogen; a hydrogenation product obtainable from a natural phospholipid, such as hydrogenated soy phosphatidyl choline; a synthetic phospholipid such as dicetyl phosphate, distearoylphosphatidylcholine, dipalmitoylphosphatidylcholine, dipalmitoylphosphatidyl glycerol, distearoylphosphatidyl glycerol, dilaurylphosphatidylglycerol, dipalmitoylphosphatidylethanolamine, dipalmitoylphosphatidylserine, eleostearoylphosphatidylcholine, eleostearoylphosphatidylethanolamine, eleostearoylphosphatidylserine, dipalmitoylphosphatidyl acid, dipalmitoylphosphatidyl ethanolamine; their salts and/or the corresponding distearoyl- and dimyristyl-counterparts; and any combination thereof.

8. The pharmaceutical composition according to claim wherein the liposomes further comprise: dicetylphosphate; cholesterol; coprostanol; cholestanol; cholestane; ergosterol; phytosterol; sitosterol; lanosterol; protein, such as albumin, immunoglobulin, casein, insulin, hemoglobin, lysozyme, immunoglobulin, [alpha]-2-macroglobulin, fibronectin, vitronectin, fibrinogen, lipase, or enzyme; α-tocopherol; stearic acid; BHT (butylhydroxytoluene); ascorbic acid; deferoxime mesylate; stearyl amine; and any combination thereof.

9. The pharmaceutical composition according to claim 5 comprising a polymer matrix, wherein the antimycotic agent is dispersed in the polymer matrix.

10. The pharmaceutical composition according to claim 9, wherein the polymer matrix comprises a gelling agent.

11. The pharmaceutical composition according to claim 9, wherein the polymer matrix comprises a film forming polymer.

12. The pharmaceutical composition according to claim 1 having a pH in the range of from 4.0 to 6.0.

13. The pharmaceutical composition according to claim 1, which is in the form of a gel formulation comprising liposomes and a polymeric matrix, wherein tenofovir is encapsulated in the liposomes and ciclopirox is dispersed in the polymer matrix.

14. The pharmaceutical composition according to claim 1, which is in the form of a spray formulation comprising liposomes and a polymeric matrix, wherein tenofovir is encapsulated in the liposomes and ciclopirox is dispersed in the polymer matrix.

15. The pharmaceutical composition according to claim 6, wherein tenofovir and/or ciclopirox are encapsulated in the liposomes.

16. The pharmaceutical composition according to claim 4 optionally further comprising a propellant selected from: a volatile hydrocarbon such as butane, propane, isobutene; or a fluorohydrocarbon (HFCs) propellant such as 1,1,1,2-tetrafluorethane, and 1,1,1,2,3,3,3-heptafluoropropane, 1,1-difluoro ethane and 1,1,1,3,3,3-hexafluoropropane, and HFC 134a; of any combination thereof.

17. A method of manufacturing a topical pharmaceutical gel composition, comprising the steps of:
(a) Dissolving an antiviral agent in a lipid core component with suitable organic solvent to obtain a solution;
(b) Homogenizing the solution of step (a) solution with water;
(c) Introducing an antimycotic agent, a film forming polymer and a preservative in water, followed by pH adjustment, to form a slurry; and
(d) Forming a gel by adding and stirring the homogenized solution with the slurry.

18. A method of manufacturing a topical pharmaceutical gel composition, comprising the steps of:
(a) Dissolving an antiviral agent in a lipid core component with suitable organic solvent to obtain a solution; and further homogenizing the solution with water;
(b) Dissolving an antimycotic agent in a lipid core component with suitable organic solvent to obtain a solution; and further homogenizing the solution with water;
(c) Introducing a film forming polymer and a preservative in water, followed by pH adjustment, to form a slurry; and
(d) Forming a gel by adding and stirring the homogenized solutions of steps (a) and (b) with the slurry.

19. A method of manufacturing a topical pharmaceutical foam composition, comprising the steps of:
(a) Dissolving a fatty alcohol and a surfactant in a suitable organic solvent to form a solution;
(b) Adding an antimycotic agent, an antiviral agent and an emollient/humectant to the solution; and
(c) Filling a canister with the solution, and pressurizing the canister with a propellant.

20. A method of manufacturing a topical pharmaceutical spray composition, comprising the steps of:
(a) Dissolving antiviral agent, soya lecithin and copolymer of N-vinylpyrrolidone-vinyl acetate in ethanol and then adding ciclopirox to it; and
(b) Filling the above solution in aluminium canisters and pressurizing with propellant.

21. A method of manufacturing a topical pharmaceutical spray composition, comprising the steps of:
(a) Dissolving antiviral agent, soya lecithin and copolymer of N-vinylpyrrolidone-vinyl acetate in ethanol;
(b) Dissolving mycotic agent, soya lecithin and copolymer of N-vinylpyrrolidone-vinyl acetate in ethanol; and
(c) Combining and filling the above solutions of step (a) and (b) in aluminium canisters and pressurizing with propellant.
